(19) United States
(12) Patent Application Publication
(10) Pub. No.: US 2011/0033418 A1
(43) Pub. Date: Feb. 10, 2011

(54) TREATMENT OF DISEASES CAUSED BY VIRAL INFECTION

(76) Inventor: Bo Niklasson, Kalmar (SE)

Correspondence Address: KOHN & ASSOCIATES, PLLC, 30500 NORTHWESTERN HWY, SUITE 410, FARMINGTON HILLS, MI, 48334, US

(21) Appl. No.: 12/817,247

(22) Filed: Jun. 17, 2010

(30) Foreign Application Priority Data
Aug. 10, 2009 (GB) 0913944.5

(51) Int. Cl. A61K 38/21; A61K 39/42; A61K 31/7056; A61P 25/00; A61P 7/06; A61P 3/10

(52) U.S. Cl. 424/85.4; 424/159.1; 514/43

(57) ABSTRACT

A method of prophylactic and/or therapeutic treatment of a mammal is provided for treating a disease that is caused by a Ljungan virus infection. The method comprises administration to the mammal of an antivirally effective amount of ribavirin or a derivative thereof effective against the Ljungan virus and pleconaril or a derivative thereof effective against the Ljungan virus to eliminate or inhibit proliferation of said virus in said mammal and at the same time prevent and/or treat said disease in said mammal. Also provided is a composition comprising ribavirin or a derivative thereof effective against a Ljungan virus and pleconaril or a derivative thereof effective against a Ljungan virus and a kit relating to this composition. Further, a method is provided for treating Ljungan virus infection comprising administering ribavirin or a derivative thereof effective against the Ljungan virus as well as a composition and a kit comprising ribavirin.

### CROSS-RELATED REFERENCE SECTION

This application claims the benefit of priority of Great Britain Patent Application Serial No. 0913944.5, filed Aug. 10, 2009, incorporated herein by reference.

### BACKGROUND OF THE INVENTION

(1) Field of the Invention

The invention relates to the treatment of diseases caused by viral infection. More precisely, the invention relates to a method of prophylactic and/or therapeutic treatment of a mammal for a disease. The disease may be caused by viral infection in most tissues or cell types. For example, the disease may be in muscle tissues, neural cells, or endocrine glands caused by a Ljungan virus infection. The invention also relates to the composition comprising ribavirin or a derivative thereof effective against a Ljungan virus and pleconaril or a derivative thereof effective against a Ljungan virus for treating a disease. The disease may be caused by viral infection in most tissues or cell types. For example, the disease may be in muscle tissues, neural cells, or endocrine glands caused by a Ljungan virus infection.

(2) Description of Related Art

Rodents are well-known reservoirs and vectors for viruses causing disease in humans. Puumala virus causing Nephropathia Epidemica (Myhrman, Nordisk Medicinsk Tidskrift, 7, 739-794, 1934; and Niklasson et al., Lancet, 1, 1012-3, 1984) is one example of an important human pathogen carried by bank voles. It has been demonstrated that the incidence rate of human Nephropathia Epidemica correlates with the vole population density during the previous year (Niklasson et al., Am. J. Trop. Med. Hyg., 53, 134-40, 1995). More recently, statistical evidence suggests that type 1 diabetes in humans also tracks the 3- to 4-year population density cycles of the bank vole with a similar time lag (Niklasson et al., Emerg. Infect. Dis., 4, 187-93, 1998). It was also observed that a high frequency of bank voles trapped in the wild and kept in the laboratory for studies of stereotypic behavior (Schoenecker et al., Appl. Anim. Behav. Sci., 68, 349-357, 2000) develop polydipsia and glucosuria at a high frequency.

Ljungan virus (LV), belonging to the Picornavirus family, is carried by small rodents and causes diseases in other animals, including humans. The first three isolates were disclosed in the International Patent Application WO 98/11133 and the partial sequence of each were also comprised therein. The full sequences of these three Ljungan viruses have also been published (Johnsson S. et al., Journal of Virology, September 2002, p. 8920-8930). A further Ljungan virus is described in Tolf et al. Journal of General Virology (2009), 90, p. 843-853.

Ljungan Virus Definition

Ljungan virus is a virus carried by small rodents. Ljungan virus belongs to the Picornavirus family. Ljungan virus is serologically and genetically distantly related to other members of the Picornavirus family. Ljungan virus will most likely form a new genus in the Picornavirus family.

Genetically the Ljungan virus genome and the polyprotein encoded by them exhibit several exceptional features, such as the absence of a predicted maturation cleavage of VP0, a conserved sequence determinant in VP0 that is typically found in VP1 of other Picornaviruses, and a cluster of two unrelated 2A proteins. The 2A1 protein is related to the 2A protein of cardio, erbo and aphthoviruses and the 2A2 protein is related to the 2A protein of parechoviruses, kobuviruses and avian encephalomyelitis virus. (A. M. Lindberg and Susanna Johansson, Virus Research 85 (2002) 61-70).

Ljungan virus is characterized by a chronic or long lasting infection in its rodent host and reservoir.

Ljungan virus can replicate and cause disease in a very broad host spectrum of animal species as well as in humans.

Ljungan virus infects these different species of animals as well as humans and the infection does often result in a long lasting or chronic infection.

Ljungan virus replicates in a wide variety of tissue culture cells giving a chronic infection with discrete CPE (cytopathogenic effect) and low viral output (in the order of 1,000-100,000 viral particles per ml supernatant).

Data generated by virus cultivation under laboratory conditions show that Ljungan virus grows/replicates in a number of cell lines that originate from different tissues and different species, e.g. Vero monkey kidney; Vero E6 monkey kidney; MA-104 monkey kidney; CV-1 monkey kidney; GMK monkey kidney; A-549 human lung; Hela human cervical tissue; BHK 21 hamster kidney; RD human muscle; and L-cells mouse skin.

In living animals and humans, Ljungan virus replicates in muscle tissue including heart tissue, in neural cell including the brain, in endocrine glands including the beta cells of the pancreas, the thyroid gland, the supra renal gland.

Data generated by detection of virus by Ljungan virus specific immunohistochemistry test, thin section electron microscopy and by PCR in humans, bank voles, lemmings, laboratory mice rabbits, guinea pigs, arctic foxes, and moose show that Ljungan virus has been found in endocrine and exocrine pancreas tissue, in endothelial cells of vessels, cells in the brain (including nerve tissue), cells of the liver, cells of the placenta and the umbilical cord, muscle tissue, heart tissue, tissue of the thyroid gland.

The conclusion is that Ljungan virus can grow in most cell types of the body and therefore infect all organs of the body.

The only group of viruses that partly interferes with this definition are viruses in the cardio virus genus.

Similarities

Cardioviruses belong to the picornavirus family.

Cardiovirus have rodents as their natural reservoir.

Cardioviruses can cause disease in a wide variety of animal species.

Cardiovirus can infect and cause disease in the same organs as Ljungan virus.

Differences

Cardiovirus and Ljungan virus are genetically distantly related.

The double 2A gene of Ljungan virus is absent in cardiovirus.

Cardiovirus is not related to Ljungan virus by serology.

Cardiovirus cause an acute disease (not long lasting or chronic) when it affects its non-rodent victims.

Cardiovirus is easy to cultivate in tissue culture without adaptation while Ljungan virus is often impossible to cultivate without blind passage in tissue culture or first passage in suckling mice and after several passages in suckling mice adaptation in tissue culture.

Cardioviruses do not infect humans (only rare case reports in the literature).

New variants of Ljungan virus have been found in different continents. They are all carried by small rodents and they all cause the same disease syndromes in humans.

How to Discover/Isolate New Strains of Ljungan Virus

The source for virus isolation/discovery can be selected/identified in different ways:

1) You may look for a wild rodent such as a mouse, rat or a vole with signs and symptoms similar to the diseases linked with Ljungan virus in humans (e.g. diabetes or myocarditis);

2) You may screen large numbers of wild rodents by PCR using several different primer combinations targeting the conserved region of the Ljungan virus genome; and

3) You may screen a large number of wild rodents using specific antisera. Antisera are collected from patients with the disease in humans linked with Ljungan virus that are living in the same geographic area as the rodents. Ljungan virus infected rodents are identified by immunostaining (e.g. immunohistochemistry) of formalin fixed organs. A portion of the same organ that is tested by immunohistochemistry is kept without fixation in a minus 70° C. freezer. The unfixed material is used for virus isolation if the immunohistochemistry turns out to be positive.

Tissue, from which virus isolation attempt will be made, is grinded and diluted in sterile saline or PBS. One-day old suckling mice are injected with 2-4 microliters of the tissue suspension intracerebrally.

When suckling mice are used for virus isolation, in general all the mice die within a week of inoculation if a virus is present. However, Ljungan virus is different in that you have to wait 10 days to 3 weeks before signs of symptoms in the baby mice develop. Signs and symptoms are very discrete such as slow weight increase and altered mobility. Only 5-10% of the animals develop symptoms. This is very unusual and would in most cases result in a negative interpretation of the isolation attempt.

Only the brain tissue from suckling mice with signs and symptoms are used for passage in new one-day old suckling mice.

When passed, the brains from sick suckling mice are grinded and diluted in sterile saline or PBS. One-day old suckling mice are injected with 2-4 microliters of the tissue suspension intracerebrally.

Several such passages may be necessary before disease develops earlier (8-12 days) and in the majority of mice.

After several passages in suckling mice Ljungan virus is inoculated into tissue culture such as Vero cells for amplification and identification.

Ljungan virus must be adapted to cell culture by passages of the cells. No or very discrete cytopathogenic effect is seen. The cells (not the tissue culture fluid) are passed weekly into new tissue culture bottles at a rate of 1 to 5. After 3-6 such blind passages the cells are stained using antibodies directed to the isolate. These antisera can be made by immunising adult mice with the suckling mouse brain suspension of a suspected isolate and/or by using human serum from patients with the disease caused by Ljungan virus living in the same geographic region as the animals used as source for virus isolation.

Previously, Ljungan virus infections have been treated using the antiviral agent pleconaril as described in U.S. Pat. No. 7,442,380 and EP 04712791.

### BRIEF SUMMARY OF THE INVENTION

The present invention provides a method of prophylactic and/or therapeutic treatment of a mammal for treating a disease that is caused by a Ljungan virus infection, comprising administration to the mammal of an antivirally effective amount of ribavirin or a derivative thereof effective against the Ljungan virus and pleconaril or a derivative thereof effective against the Ljungan virus to eliminate or inhibit proliferation of said virus in said mammal and at the same time prevent and/or treat said disease in said mammal.

The present invention also provides a composition comprising ribavirin or a derivative thereof effective against a Ljungan virus and pleconaril or a derivative thereof effective against a Ljungan virus and a kit relating to this composition.

The present invention further provides a method for treating Ljungan virus infection comprising administering ribavirin or a derivative thereof effective against the Ljungan virus as well as a composition and a kit comprising ribavirin.

### BRIEF DESCRIPTION OF THE SEVERAL VIEWS OF THE DRAWING

Other advantages of the present invention will be readily appreciated as the same becomes better understood by reference to the following detailed description when considered in connection with the accompanying drawing wherein:

FIG. 1 is a graph showing the age at diabetes onset in diabetes prone BB rats.

### DESCRIPTION OF THE INVENTION

The present invention is directed to a method of prophylactic and/or therapeutic treatment of a mammal for a disease that is caused by a Ljungan virus infection, comprising administration to said mammal of an antivirally effective amount of ribavirin or a derivative thereof effective against the Ljungan virus and pleconaril or a derivative thereof effective against the Ljungan virus to eliminate or inhibit proliferation of said virus in said mammal and at the same time prevent and/or treat said disease in said mammal.

It has been found that using the combination of pleconaril and ribavirin to prevent/treat Ljungan virus infection provides better results than using either compound on its own.

Preferably the mammal is selected from the group consisting of humans, horses, cattle, pigs, cats, dogs and rodents such as rats, mice and bank voles.

The disease caused by Ljungan virus infection may be caused by the infection of a tissue or cell type. As indicated above, it is known that Ljungan virus is capable of growth in most cell types of the body and can therefore infect all organs of the body.

In a preferred embodiment of the invention, the Ljungan virus infection is in at least one of muscle tissues, neural cells and endocrine glands of the mammal, and the infection may also be in an organ, such as the liver or pancreas.

In another embodiment the muscle tissue is heart tissue, the neural cells are brain cells, the endocrine glands are beta cells of pancreas, thyroid gland and/or supra renal gland.

In yet another embodiment the disease is selected from the group consisting of myocarditis, cardiomyopathia, Guillain Band syndrome, diabetes mellitus, multiple sclerosis, chronic fatigue syndrome, myasthenia gravis, amyothrophic lateral sclerosis, dermatomyositis, polymyositis, malformation such as anencephaly and hydrocephaly, spontaneous abortion, intrauterine fetal death, pre-eclampsia, lethal central nervous disease, sudden infant death syndrome, Bell's (facial) paralysis, Addison's disease and pernicious anemia. Preferably, the disease is selected from diabetes mellitus and amyothrophic lateral sclerosis.

In one embodiment, the method further comprises administering an anti-Ljungan virus antibody. This is an antibody which can neutralise the Ljungan virus and can be obtained, for example, from patients who have been infected with Ljungan virus, as described above. Preferably, the method comprises administering antiserum which is effective against the Ljungan virus.

In another embodiment, the method further comprises administering an interferon, for example, interferon-γ.

The chemical name of pleconaril is (3-[3,5-dimethyl-4-[[3-(3-methyl-5-isoxazolyl)propyl]oxy]phenyl]-5-(trifluoromethyl)-1,2,4-oxadiazole) (ViroPharma Inc.; Picovir®).

The structural formula of pleconaril is:

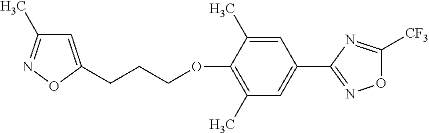

Compounds comprising pleconaril and derivatives thereof are shown in U.S. Pat. No. 5,464,848, the European equivalent EP 0566199, and are claimed to have effect on picornaviral infections.

Other derivatives of pleconaril claimed to have antiviral effects against picornavirus are disclosed e.g. in U.S. Pat. No. 4,945,164. Therefore, compounds which are derivatives of pleconaril would be apparent to a skilled person who would easily be able to identify them as such.

The chemical name of ribavirin is 1-[(2R,3R,4S,5R)-3,4-dihydroxy-5-(hydroxymethyl)oxolan-2-yl]-1H-1,2,4-triazole-3-carboxamide and ribavirin is sold under the brand names Copegus™, Rebetol™, Ribasphere™, Vilona™ and Virazole™.

The structural formula of ribavirin is:

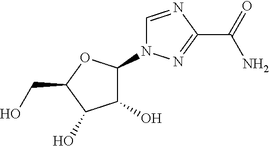

Derivatives of ribavirin are well known to those skilled in the art and include, for example, taribavirin (1-[(2R,3R,4S,5S)-3,4-dihydroxy-5-(hydroxymethyl)oxolan-2-yl]-1,2,4-triazole-3-carboximidamide), mizoribine and aminoimidazole carboxamide ribonucleotide (AICAR). Derivatives of ribavirin are disclosed, for example, in U.S. Pat. No. 3,897,415.

Candidate compounds can be tested by use of animal models or in vitro infected cell or tissue cultures in a way known to a man skilled in the art, e.g. as shown in the Examples below and as described in U.S. Pat. No. 7,442,380 and EP 04712791.

All the mentioned references are incorporated herein by reference.

Another aspect of the invention is directed to a composition of ribavirin or a derivative thereof effective against a Ljungan virus and pleconaril or a derivative thereof effective against a Ljungan virus which can be used for treating a disease in a mammal that is caused by a Ljungan virus infection.

Further, the invention also provides the use of the combination of ribavirin or a derivative thereof effective against a Ljungan virus and pleconaril or a derivative thereof effective against a Ljungan virus for the preparation of a medicament for the treatment of a disease in a mammal that is caused by a Ljungan virus infection.

Preferably the mammal is selected from the group consisting of humans, horses, cattle, pigs, cats, dogs and rodents such as rats, mice and bank voles.

The disease caused by Ljungan virus infection may be caused by the infection of any tissue or cell type. As indicated above, it is known that Ljungan virus is capable of growth in most cell types of the body and can therefore infect all organs of the body.

In a preferred embodiment of the invention the Ljungan virus infection is in at least one of muscle tissues, neural cells and endocrine glands of the mammal, but the infection may also be in an organ, such as the liver or pancreas.

In another embodiment of this aspect of the invention the muscle tissue is heart tissue, the neural cells are brain cells, the endocrine glands are beta cells of pancreas, thyroid gland and/or supra renal gland.

In yet another embodiment the disease is selected from the group consisting of myocarditis, cardiomyopathia, Guillain Barré syndrome, diabetes mellitus, multiple sclerosis, chronic fatigue syndrome, myasthenia gravis, amyothrophic lateral sclerosis, dermatomyositis, polymyositis, malformation such as anencephaly and hydrocephaly, spontaneous abortion, intrauterine fetal death, pre-eclampsia, lethal central nervous disease, sudden infant death syndrome, Bell's (facial) paralysis, Addison's disease and pernicious anemia. Preferably, the disease is selected from diabetes mellitus and amyothrophic lateral sclerosis.

In one embodiment, the composition may further comprise an anti-Ljungan virus antibody. Preferably, the composition comprises an antiserum which is effective against Ljungan virus.

In another embodiment, the composition may further comprise an interferon, for example, interferon-γ.

Medicament

While it is possible that the antiviral compounds may be administered as the neat chemical, it is preferable to present the active ingredients as a pharmaceutical composition, formulation or as a medicament.

The ribavirin or a derivative thereof and pleconaril or a derivative thereof may be present in the same dosage form or may be present in separate dosage forms. If present in the same dosage form, the ribavirin or a derivative thereof and pleconaril or a derivative thereof may be formulated together or may be present in separate compartments of a multi-compartment dosage form, such as a multi-layer tablet, or a compartmentalised capsule.

In one embodiment, the invention comprises a composition comprising ribavirin or a derivative thereof effective against a Ljungan virus and pleconaril or a derivative thereof effective against a Ljungan virus.

A suitable composition, medicament or pharmaceutical formulation useful in the present invention comprises ribavirin or derivative thereof and/or pleconaril or derivative thereof together with one or more pharmaceutically acceptable carriers and, optionally, other therapeutic and/or prophylactic ingredients. The carrier(s) must be acceptable in the sense that it should be compatible with the other active or inactive ingredients of the formulation and not deleterious to the recipient thereof.

The antiviral compounds used in the invention may also be used in combination with other anti-viral agents or other pharmaceuticals used in the treatment of viral infections.

Representative examples of other active ingredients include immunomodulators, immunostimulants, such as various interleukins, cytokines and antibody preparations, antibiotics and anti-inflammatory agents.

Pharmaceutical formulations include those suitable for oral, rectal, nasal, topical (including buccal and sub-lingual), vaginal or parenteral (including intramuscular, subcutaneous and intravenous) administration or in a form suitable for administration by inhalation or insufflation. The compounds of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The invention also provides a composition comprising ribavirin or a derivative thereof effective against a Ljungan virus and pleconaril or a derivative thereof effective against a Ljungan virus for use in therapy.

Further, the invention provides a composition comprising ribavirin or a derivative thereof effective against a Ljungan virus and pleconaril or a derivative thereof effective against a Ljungan virus for use in treating a disease caused by Ljungan virus.

Therapy

Antiviral Compounds

Ribavirin or a derivative thereof and pleconaril or a derivative thereof are in many cases the only active ingredients needed for termination of Ljungan virus infection and thereby reverse the disease in muscle tissues, neural cells, or endocrine glands that is caused by a Ljungan virus infection.

The ribavirin or derivative thereof and pleconaril or derivative thereof can be administered to the mammal simultaneously with each other or they can be administered sequentially. When the ribavirin or derivative thereof and pleconaril or derivative thereof are administered simultaneously, they may be administered together in one composition or simultaneously in two separate compositions.

Interferon

It is very likely that Ljungan virus is interferon sensitive. It can be expected that interferon can have an effect if given before the infection or very early in the course of infection. It is very likely that interferon can be used in combination with other therapeutic measures to terminate a chronic infection. The combination can be immunoglobulins, vaccination and antiviral compounds. This can be evaluated in an animal model.

Antibody

Data from picornavirus infection suggests that specific antibodies can be used in the treatment of severe and chronic infections. It can be used by itself or in combination with other therapeutic measures. Antibodies to Ljungan virus can be as polyclonal antibodies prepared from immune humans or animals or as human monoclonal antibodies or as “humanized” monoclonal antibodies originating from an animal system such as mice. The Ljungan virus antibodies can be used in combination with the antiviral compounds and possibly interferon.

The present invention also provides a method of prophylactic and/or therapeutic treatment of a mammal for a disease that is caused by a Ljungan virus infection, comprising administration to said mammal a composition comprising ribavirin or a derivative thereof effective against the Ljungan virus to a mammal receiving pleconaril or a derivative thereof effective against the Ljungan virus simultaneously or sequentially with the pleconaril or a derivative thereof.

Further, the present invention provides a method of prophylactic and/or therapeutic treatment of a mammal for a disease that is caused by a Ljungan virus infection, comprising administration to said mammal a composition comprising pleconaril or a derivative thereof effective against the Ljungan virus to a mammal receiving ribavirin or a derivative thereof effective against the Ljungan virus simultaneously or sequentially with the ribavirin or a derivative thereof.

Additionally, the present invention provides a kit comprising a first pharmaceutical composition comprising ribavirin or a derivative thereof effective against a Ljungan virus and a second pharmaceutical composition comprising pleconaril or a derivative thereof effective against a Ljungan virus, the first and second pharmaceutical compositions being suitable for simultaneous or sequential administration for the treatment of a disease that is caused by a Ljungan virus infection.

In one embodiment, the kit further comprise a third pharmaceutical composition comprising an anti-Ljungan virus antibody. Preferably, the kit comprises an antiserum which is effective against the Ljungan virus.

In another embodiment, the kit further comprises a third (or fourth) pharmaceutical composition comprising an interferon, for example, interferon-γ.

In another aspect, the present invention is directed to a method of prophylactic and/or therapeutic treatment of a mammal for a disease that is caused by a Ljungan virus infection, comprising administration to said mammal of an antivirally effective amount of ribavirin or a derivative thereof effective against the Ljungan virus to eliminate or inhibit proliferation of said virus in said mammal and at the same time prevent and/or treat said disease in said mammal.

Preferably the mammal is selected from the group consisting of humans, horses, cattle, pigs, cats, dogs and rodents such as rats, mice and bank voles.

In a preferred embodiment, the Ljungan virus infection is in at least one of muscle tissues, neural cells and endocrine glands of the mammal, and the infection may also be in an organ, such as the liver or pancreas.

In another embodiment the muscle tissue is heart tissue, the neural cells are brain cells, the endocrine glands are beta cells of pancreas, thyroid gland and/or supra renal gland.

In yet another embodiment the disease is selected from the group consisting of myocarditis, cardiomyopathia, Guillain Barré syndrome, diabetes mellitus, multiple sclerosis, chronic fatigue syndrome, myasthenia gravis, amyothrophic lateral sclerosis, dermatomyositis, polymyositis, malformation such as anencephaly and hydrocephaly, spontaneous abortion, intrauterine fetal death, pre-eclampsia, lethal central nervous disease, sudden infant death syndrome, Bell's (facial) paralysis, Addison's disease and pernicious anemia. Preferably, the disease is selected from diabetes mellitus and amyothrophic lateral sclerosis.

In one embodiment, the method further comprises administering an anti-Ljungan virus antibody. Preferably, the method comprises administering antiserum which is effective against the Ljungan virus.

In another embodiment, the method further comprises administering an interferon, for example, interferon-γ.

Another aspect of the invention is directed to a composition of ribavirin or a derivative thereof effective against a Ljungan virus which can be used for treating a disease in a mammal that is caused by a Ljungan virus infection. More specifically, the composition comprises: a) ribavirin or a derivative thereof effective against a Ljungan virus; and b) one or more agents selected from the group consisting of an anti-Ljungan virus antibody and an interferon.

Preferably, the composition comprises an antiserum which is effective against Ljungan virus.

The interferon may be, for example, interferon-γ.

Additionally, the present invention provides a kit comprising a first pharmaceutical composition comprising ribavirin or a derivative thereof effective against a Ljungan virus and a second pharmaceutical composition comprising an anti-Ljungan virus antibody, the first and second pharmaceutical compositions being suitable for simultaneous or sequential administration for the treatment of a disease that is caused by a Ljungan virus infection.

The present invention also provides a kit comprising a first pharmaceutical composition comprising ribavirin or a derivative thereof effective against a Ljungan virus and a second pharmaceutical composition comprising an interferon, the first and second pharmaceutical compositions being suitable for simultaneous or sequential administration for the treatment of a disease that is caused by a Ljungan virus infection.

In one embodiment, the kit comprises a first pharmaceutical composition comprising ribavirin or a derivative thereof effective against a Ljungan virus, a second pharmaceutical composition comprising an interferon, and a third pharmaceutical composition comprising an anti-Ljungan virus antibody, the first, second and third pharmaceutical compositions being suitable for simultaneous or sequential administration for the treatment of a disease that is caused by a Ljungan virus infection.

Preferably, the kit comprises an antiserum which is effective against the Ljungan virus.

The interferon may be, for example, interferon-γ.

As will be appreciated by a person skilled in the art, much of the information relating to the combination of pleconaril' or a derivative thereof effective against a Ljungan virus and ribavirin or a derivative thereof effective against a Ljungan virus is equally applicable to the use of ribavirin or a derivative thereof effective against a Ljungan virus on its own.

Determination of Presence of Ljungan Virus

The presence of Ljungan virus can be determined using any standard procedure including immunohistochemistry using antibody molecules having affinity for Ljungan virus or by using a labelled nucleic acid probe capable of specifically hybridising to Ljungan virus nucleic acid. Alternatively the presence of Ljungan virus can be determined by detecting the presence of anti-Ljungan virus antibodies using a suitable test. Suitable techniques for determining the presence of Ljungan virus or anti-Ljungan virus antibodies are described in the examples below. Determination of the presence of Ljungan virus RNA by Polymerase Chain Reaction (PCR) is also described in the examples below.

The invention will now be described in details, by way of example only with reference to the accompanying figures in which:

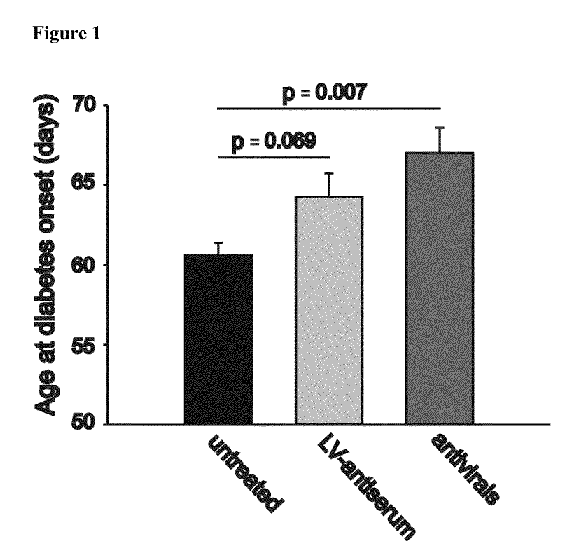

FIG. 1 shows the age at diabetes onset in diabetes prone BB rats. The prediabetic rats were untreated (black), treated with LV-antiserum (light grey) or treated with the antivirals pleconaril and ribavirin (dark grey). The untreated group is composed of the control groups for the LV-antiserum and antivirals treated groups, i.e. subjected to saline or tap water.

### Examples

Antiviral treatments delay onset of diabetes in Ljungan virus infected CD-1 mice and diabetes prone BB rats

Aims/hypothesis The influence of Ljungan virus (LV) on the development of diabetes in the BioBreeding (BB) rat, a carrier of LV and an animal model for type 1 diabetes, and the CD-1 mouse was examined.

Methods Pregnant CD-1 mice were infected with LV. The male offspring of these dams were subjected to stress and treated with LV-antiserum or antiviral drug alone, a combination of both, or left untreated as controls. Another control group was neither infected in utero, nor treated, but subjected to stress. Blood glucose, insulin levels and epididymal fat were analyzed. BB rats were given LV-antiserum, antiviral drugs or were untreated.

Results Male CD-1 mice exposed to LV in utero and a stress regime post partum developed a type 2-like diabetes. Treatment with LV-antiserum or the antiviral drug reduced the occurrence of diabetes, but only the combination therapy significantly reduced not only the levels of blood glucose, but also insulin levels and the amount of abdominal fat. In the group of diabetes prone (DP) BB rats treated with LV-antiserum, the prediabetic phase was prolonged four days and in the group treated with antiviral drugs, the onset was delayed one week.

Conclusions/interpretations Male CD-1 mice, exposed to LV in utero and stress after birth, develop diabetes that can be prevented with antiviral treatment. In BB rats treatment with antiserum or antiviral drugs delays the onset of diabetes. These results indicate that the presence of LV can be involved in the pathogenesis of diabetes in these animal models.

Introduction

The pathogenesis of type 1 diabetes includes genetic predisposition, environmental factors and dysregulation of the immune system. Among the environmental triggers, various viral infections have been proposed to initiate or facilitate the pathogenic process leading to type 1 diabetes [1-4]. It has been proposed that type 1 diabetes in humans may be caused by one (or more) infectious agents carried by rodents, based on observations of an association between rodent density and disease incidence [5]. These observations initiated a search for an etiologic agent in small rodents and it resulted in the isolation of a novel picornavirus from the bank vole (Myodes glareolus). The virus was named “Ljungan virus” after the valley in Västernorrland County in Sweden where it was first observed [6]. Bank voles express type 1 diabetes in captivity including lysis of pancreatic islet beta cells and presence of autoantibodies against GAD65-, IA-2 and insulin [7, 8]. In CD-1 mice, that are not carriers of LV, it has been shown that adult male mice develop a type 2 like diabetes if they have been infected in utero with LV and are subjected to a stress regime after birth [9].

Diabetes-prone (DP) BioBreeding (BB) rats, an animal model for type 1 diabetes, become diabetic at around 60 days of age, which gives an excellent opportunity to study the impact of different prediabetic interventions on the development of beta cell destruction and onset of diabetes. As in humans, there have been speculations that different viruses may play a role in the pathogenesis of type 1 diabetes. A virus identified as Kilham's rat virus (KRV) was isolated from diabetes resistant (DR) BB rats during an outbreak of lymphocytic insulitis and hyperglycemia in this group. When isolated and given to healthy DR rats, KRV induced diabetes. However, KRV could not be found in pancreatic islets, and beta cell cytolysis was not observed until after the appearance of lymphocytic insulitis [10]. Rat cytomegalovirus (RCMV) has been shown to shorten the prediabetic period in DP rats. The virus did not infect the pancreatic islets, but resulted in an influx of peritoneal macrophages into the pancreas [11]. We have identified LV in the pancreatic islets of BB rat colonies [12]. The virus was found in prediabetic, diabetic as well as DR rats. The aim of this study was to investigate whether the treatment of LV infection in two distinct animal models of diabetes could influence the onset of diabetes.

Subjects, Materials and Methods

Animals CD-1 (ICR) mice were obtained from Br Charles River laboratories, Germany. Glucose tolerance test was performed to diagnose diabetes. In this test 2 g glucose per kg body weight was injected intraperitoneally (as 100 mg glucose/ml solution). Blood glucose was measured (Medisence Precision PCx Abbott Laboratories) before the injection of glucose and after 60 and 120 min. Diabetes was defined as a blood glucose level above 200 mg/dL at 120 min. Abdominal fat in male mice was measured by dissecting out the bilateral scrotal fat cushion. The total weight of both fat cushions was used.

The DP BB rats were obtained from the breeding colony maintained at Karolinska Institutet. The overall incidence of diabetes among the DP rats is 100% with 60 days as the mean age of onset. The animals were housed under Specific Pathogen Free (SPF) conditions in a temperature- and humidity-controlled room with 12 hours light dark cycles. They were fed R36 diet and water ad libitum. All animals were followed until the onset of type 1 diabetes (blood glucose 270 mg/dL or above and/or ketons). Animals of both genders were used.

The studies performed in CD-1 mice and BB rats were approved by the Animal Experiment Ethics Committees at Karolinska Institutet.

Preparation of antiserum The brain from suckling mice infected intra-cerebrally at day 1 with the LV strains 145SL, 87-012 and 174F were used for immunization and infection. Antiserum to LV was made by infecting/immunizing adult mice with a 10% suckling mouse brain homogenate [6]. The immune sera were collected from the adult mice 6-8 weeks after infection and pooled.

Antiviral agents Pleconaril (VP-63843) a novel, orally bio-available, broad spectrum anti-picorna-viral agent developed as a therapeutic tool in entero- and rhino viral infections, was used alone or in combination with specific antiserum for intervention [13-15]. In tissue culture Pleconaril has been found to have some inhibitory effect on LV, but not at the same magnitude as found when testing picornaviruses known to be sensitive to the drug.

Ribavirin (1-beta-D-ribafuranosyl-1,2,4-triazole-3-carboxamide), a synthetic nucleoside analogue with activity against a wide variety of both RNA and DNA viruses [16], was used in combination with Pleconaril in the BB rat experiments.

Analysis of insulin Serum insulin was measured using a rat insulin ELISA kit (Mercodia AB).

Transmission of LV to CD-1 mice followed by antiviral treatments Pregnant outbred CD1 mice were infected intraperitoneally with 1000 ID50 (determined by intra cerebral titration in suckling mice) units of LV145 SL two days after conception. Control animals were given an injection of saline. All female mice were kept in individual cages and followed for the development of gestational diabetes, pregnancy outcome and development of diabetes in the offspring. In all the pregnant mice mild stress was induced by adding glucose (100 g/L) to drinking water, performing weekly weights and a glucose tolerance test at the end of the pregnancy. At five weeks of age antiviral therapy to the male offsprings, using LV-specific murine antiserum or the oral antiviral compound Pleconaril (200 mg per kg body weight) or a combination of both, were given once a day for four consecutive days. The control groups of infected and uninfected male offsprings were untreated. From the age of 25 days all mice were subjected to stress by placing 4-5 male mice from different litters in the same cage. At 15 weeks of age the animals were subjected to a glucose tolerance test. Serum insulin levels and abdominal fat were measured. Since it has previously been shown that female offsprings do not develop diabetes [9], they were excluded from this study.

Antiserum and antiviral treatments in BB rats A group of 12, 38 days old, DP rats were given a single dose i.p. of LV-specific murine antiserum (group I) and 13 control DP rats, of the same age, were given an injection of saline (group II).

A group of six, 38 days old, DP rats were treated with a combination of the two antiviral compounds, Pleconaril and Ribavirin (200 mg of each per kg body weight), orally with a tube connected to a syringe two times daily for nine consecutive days (group III). Five control DP rats of the same age were given equal volume of tap water for the same period of time (group IV).

Statistics The glucose tolerance test in the CD-1 mice was evaluated by Fisher's exact one sided test. Otherwise statistical significance in the CD-1 mice was evaluated by Student's t-test. P values <0.05 were considered significant. Data are presented as means±s.e.m.

Analysis of variance was used to test differences among treatments in the days-to-diabetes-onset variable in the BB rat experiment. The control groups for the antiserum and antiviral testing in the BB rat experiment were combined following an initial test. The three remaining treatments in the days-to-diabetes-onset variable were then analyzed separately. Specific pairwise differences among groups resulting from multiple comparisons testing following analysis of variance were evaluated with Bonferroni corrected tests. Data are presented as means±s.e.m.

Results

Ljungan virus in CD-1 mice LV-infected pregnant mice showed no signs of diabetes or abnormal glucose tolerance. However, surviving male offspring developed diabetes 10-15 weeks after birth. Five treatment groups of these male offspring (Table 1) were developed: I, uninfected control mice; II, LV infected mice without antiviral or antiserum treatment; III, infected mice treated with the antiviral compound Pleconaril; IV, infected mice treated with LV antiserum; and V, infected and treated with both Pleconaril and LV antiserum.

All mice were examined for hyperglycemia with a standard glucose tolerance test. Defining >200 mg/dL of blood glucose at 120 minutes as having hyperglycemia, the infected but untreated group stood apart from the other treatments with 8 out of 11 (73%) of the mice being hyperglycemic (PExact, 2×5 test <0.001). Further, the four groups either uninfected or treated (I, III, IV and V) had low or entirely absent frequency of hyperglycemia (0-14%), and formed a statistically homogeneous subgroup (P=0.61). However, only the combination therapy, including both Pleconaril and antiserum, was effective in significantly reducing the other indicators of a diabetic state. The combination therapy resulted in lower blood glucose at 60 minutes (P=0.017), lower abdominal fat (P=0.012) and reduced serum insulin (P=0.032) (using one-sided t-test comparing groups II and V).

Effects of treatments with antiserum and antiviral agents in BB-rats Four treatment groups were initially constructed for the study; I, a group receiving antiserum directed against LV; II, a group receiving saline and serving as a control for the first group; III, a group receiving a combination of the antiviral drugs Pleconaril and Ribavirin; and IV, a group receiving the same volume of drinking water as the third group. The two control groups (the second and the fourth group) were combined after they were found not to differ in days to diabetes onset (F=0.0001, P=0.99).

Single classification analysis of variance performed on the control group, control+antiserum and control+antiviral treatment groups testing for differences in days-to-diabetes was significant (F=6.48, P=0.0042). These differences were accounted for by additional days to diabetes onset for each of the two treated groups (64.3 days for the antiserum treated and 67.0 days for the antiviral treated) compared to the control group (60.6 days), see FIG. 1. This demonstrates that each of the treatments was effective in delaying the diabetes onset.

TABLE 1
The effects of Pleconaril and LV antiserum on blood glucose,
abdominal fat and serum insulin in LV infected CD1 male mice and controls with all animals
subjected to the stress regimen.

| | | No. with blood | Blood | | |
|---|---|---|---|---|---|
| | No. of | glucose >200 mg/dL | glucose at 60 min | Abdominal | Serum insulin |
| Group | mice | at 120 min | (mg/dL) | fat (g) | (μg/L) |
| I. Non-infected control | 14 | 2 (14%) | 190 ± 7.8 | 1.8 ± 0.26 | 1.09 ± 0.33 |
| II. LV only | 11 | 8 (73%)^{a} | 242 ± 22.7 | 2.9 ± 0.25 | 5.04 ± 0.73 |
| III. LV + Pleconaril | 11 | 1 (9%) | 211 ± 23.8 | 2.1 ± 0.31 | 4.15 ± 1.52 |
| IV. LV + Antisera | 10 | 0 (0%) | 210 ± 23.1 | 2.5 ± 0.35 | 6.67 ± 2.15 |
| V. LV + Pleconaril + Antisera | 10 | 0 (0%) | 181 ± 12.3^{b} | 2.1 ± 0.21^{c} | 2.86 ± 0.82^{d} |

^{a}Significantly higher than the other four treatments, which are homogeneous
^{b}Significantly different from the Untreated group (II) at P = 0.017
^{c}Significantly different from the Untreated group (II) at P = 0.012
^{d}Significantly different from the Untreated group (II) at P = 0.032

Discussion

In this study we examined the possible significance of LV in the development of diabetes in two different species, namely CD-1 mice, without a genetic disposition for diabetes, and BB rats, which are prone to develop type 1 diabetes.

Male CD-1 mice that had been exposed to LV in utero and subjected to stress after birth developed, at the age of 10-15 weeks, a type 2-like diabetes with increased blood glucose, elevated insulin levels and increased epididymal fat (Table 1). The hypothesis that psychological stress may lead to diabetes has been supported in several studies [17]. This connection has also been demonstrated in the wild bank voles, known to carry LV [6, 18]. LV exposure, in combination with stress, has also been shown to lead to diabetes in male CD-1 mice [9] and intrauterine death in pregnant CD-1 mice [19].

The mothers of the offspring, used in this study, were subjected to both stress (including glucose in the drinking water, weekly weighings and glucose tolerance tests) and viral exposure during pregnancy, but they did not develop diabetes. After birth the mice were transferred to cages with other mice. Mixing the litters seems particularly stressful on male mice as they got diabetes, which is in contrast to the female mice that did not get diabetes. These observations suggest that the LV infection by itself is not enough to disturb the beta cell function, but a component of stress is essential too. Furthermore, females seem to be less sensitive to stress, as they do not develop diabetes, although they are exposed to the virus.

It has previously been shown that captured wild bank voles develop type 1 diabetes [8], while bank voles born in captivity develop type 2 diabetes [20]. In line with these results, it has been reported that encephalomyocarditis (EMC) virus causes type 1 diabetes [21] as well as type 2 diabetes [22]. Hence, a single species of virus can cause both type 1 and type 2 diabetes in the same species. Mice infected with LV develop a type 2-like illness regardless of the stress level, while the bank voles can develop both type 2 diabetes and type 1 diabetes. It is possible that the combination of virus strain and the genetic background of the infected individual result in either a type 1- or a type 2-like disease and that both disease outcomes never occur in the same individual. It is also possible that an individual animal first develops a type 2-like illness and that the same animal depending on genetic susceptibility and stress level later can develop a type 1-like disease.

In BB rats, LV has been found in both DR and prediabetic and diabetic DP rats [12]. To evaluate if the presence of LV has any influence on the onset of type 1 diabetes, 38 days old prediabetic DP rats were treated with a single i.p. dose of an antiserum against LV or a combination of two antiviral drugs for nine days. The prolongation of the prediabetic phase was about one week and since our colony of DP rats normally develop type 1 diabetes within a very narrow time range, and taking into account the lifespan of a rat, this delay is of biological significance. The fact that we could not completely inhibit the onset of diabetes might be due to several reasons. Two major concerns are whether the timing of the treatment was optimal and whether the antiviral treatment was effective in eradicating the virus. The antiviral agents used are not specific for LV (although Pleconaril is specific for the Picornavirus family, including LV), and presently there are no LV-specific drugs available. Elimination of LV after treatment could not be investigated, as it is not advisable to take biopsies from the pancreas in a living animal.

There are previous reports regarding virus and diabetes in the BB rat. KRV has been shown to reproducibly induce diabetes in DR rats, but do not affect the onset of type 1 diabetes in DP rats [10]. RCMV infection, on the other hand, shortened the prediabetic period in DP rats, but did not induce diabetes in DR rats[23]. In our colony, LV has been identified in the islets of both categories of BB rats, and treatments aiming to remove the virus in the DP rats prolonged the prediabetic period, but on the other hand, none of our DR rats acquired type 1 diabetes despite being carriers of LV. Hence, there is a complicated, and still not fully understood, interplay between environmental agents, the immune system and the genetic background in the development of type 1 diabetes.

Our results show that the combination of ribavirin and pleconaril delay the onset of diabetes in BB rats. This combination has been shown to be significantly better in preventing diabetes than either of the two antiviral compounds on their own.

Our results, that LV antiserum and antiviral drug treatment reduces or delays the onset of diabetes in in utero infected CD-1 mice and DP BB rats that are carriers of the virus, indicate that the presence of LV can play a role in the development of diabetes.

Treatment of Amyotrophic Lateral Sclerosis (ALS)

ALS is a progressive neurodegenerative disease that primarily involves the motor neuron system. The disease is of unknown origin. Several genetic components have been identified. An environmental factor is also believed to play a role. The inventors believe that Ljungan virus (LV) in combination with a genetic pre disposition causes ALS.

ALS in Humans

Brain stem (medulla oblongata) tissue from autopsy material from 10 ALS patients have been investigated by immunohistochemistry (IHC). Eight of the 10 patients were found positive.

The IHC was performed as follows:

Formalin fixed paraffin embedded organs (brain) were analyzed from the 10 ALS cases. Presence of LV specific antigen was visualized by two different LV specific mouse monoclonal antibodies. Normal mouse serum was used as a control for the monoclonal antibodies. In addition, LV capsid protein were stained using a rabbit antiserum against recombinant LV VP1. As a control to the rabbit VP1 antiserum we used serum from a rabbit immunized using the same protocol but with the carrier GST protein only.

ALS in Mice

An ALS mouse model was used in which the mice are transgenic for superoxide dismutase 1 (SOD1). The inventors have discovered that this mouse model (strain B6SJL-Tg-SOD1-G93) distributed by Charles River company, USA (a major animal breeder) is infected with LV. The inventors believe that the combination of LV infection and the presence of the SOD gene in these mice causes ALS. Mice uniformly develop paralysis at 90 days of age and all die before 120 days of age. The inventors treated the LV infection in these mice using two different antiviral compounds separately and in combination. One group of mice was kept as a control.

Antiviral agents Pleconaril (VP-63843) a novel, orally bio-available, broad spectrum anti-picorna-viral agent developed as a therapeutic tool in entero- and rhino viral infections, was used alone or in combination with specific antiserum for intervention. Ribavirin (1-beta-D-ribafuranosyl-1,2,4-triazole-3-carboxamide), a synthetic nucleoside analogue with activity against a wide variety of both RNA and DNA viruses, was used alone or in combination with Pleconaril.

Groups of seven 35 day old B6SJL-Tg-SOD1-G93 mice were treated with two antiviral compounds, Pleconaril and Ribavirin (200 mg of each per kg body weight), orally with a tube connected to a syringe daily for 50 consecutive days. Mice were observed every day for signs of paralysis.

The experiment was terminated at day 120 when all untreated animals were expected to have developed severe paralysis and death.

TABLE 2

| | No. | | Mice with | Mice |
|---|---|---|---|---|
| | of | | paralysis | without |
| Group | Mice | Therapy | (including death) | paralysis |
| A | 7 | Pleconaril | 5 | 2 |
| B | 7 | Ribavirin | 6 | 1 |
| C | 7 | Ribavirin and Pleconaril | 0 | 7 |
| D | 7 | No therapy (control) | 7 | 0 |

### REFERENCES

[1] Gamble D R, Kinsley M L, FitzGerald M G, Bolton R, Taylor K W (1969) Viral antibodies in diabetes mellitus. Br Med J 13; 3: 627-630
[2] Forrest J M, Menser M A, Harley J D (1969) Diabetes mellitus and congenital rubella. Pediatrics 44: 445-447
[3] Yoon J W, Austin M, Onodera T, Notkins A L (1979) Isolation of a virus from the pancreas of a child with diabetic ketoacidosis. N Engl J Med 24; 300: 1173-1179
[4]Härkönen T, Paananen A, Lankinen H, Hovi T, Vaarala O, Roivainen M (2003) Enterovirus infection may induce humoral immune response reacting with islet cell autoantigens in humans. J Med Virol 69: 426-440
[5] Niklasson B, Hörnfeldt B, Lundman B (1998) Could myocarditis, insulin-dependent diabetes mellitus, and Guillain-Barré syndrome be caused by one or more infectious agents carried by rodents? Emerg Infect Dis 4: 187-193
[6] Niklasson B, Kinnunen L, Hörnfeldt B, et al. (1999) A new picornavirus isolated from bank voles (Clethrionomys glareolus). Virology 255: 86-93
[7] Niklasson B, Hörnfeldt B, Nyholm E, et al. (2003) Type 1 diabetes in Swedish bank voles (Clethrionomys glareolus): signs of disease in both colonized and wild cyclic populations at peak density. Ann N Y Acad Sci 1005: 170-175
[8] Niklasson B, Heller K E, Schønecker B, et al. (2003) Development of type 1 diabetes in wild bank voles associated with islet autoantibodies and the novel Ijungan virus. Int J Exp Diabesity Res 4: 35-44
[9] Niklasson B, Samsioe A, Blixt M, et al. (2006) Prenatal viral exposure followed by adult stress produces glucose intolerance in a mouse model. Diabetologia 49: 2192-2199
[10] Guberski D L, Thomas V A, Shek W R, et al. (1991) Induction of type I diabetes by Kilham's rat virus in diabetes-resistant BB/Wor rats. Science 15; 254: 1010-1013
[11] Hillebrands J L, van der Werf N, Klatter F A, Bruggeman C A, Rozing J (2003) Role of peritoneal macrophages in cytomegalovirus-induced acceleration of autoimmune diabetes in BB-rats. Clin Dev Immunol 10: 133-139
[12] Niklasson B, Hultman T, Kallies R, et al. (2007) The BioBreeding rat diabetes model is infected with Ljungan virus. Diabetologia 50: 1559-1560
[13] Florea N R, Maglio D, Nicolau D P (2003) Pleconaril, a novel antipicornaviral agent. Pharmacotherapy 23 339-348
[14] Romero J R (2001) Pleconaril: a novel antipicornaviral drug. Expert Opin Investig Drugs 10: 369-379
[15] Pevear D C, Tull T M, Seipel M E, Groarke J M (1999) Activity of pleconaril against enteroviruses. Antimicrob Agents Chemother 43: 2109-2115
[16] Graci J D, Cameron C E (2006) Mechanisms of action of ribavirin against distinct viruses. Rev Med Virol 16: 37-48
[17] Sepa A, Ludvigsson J (2006) Psychological stress and the risk of diabetes-related autoimmunity: a review article. Neuroimmunomodulation 13: 301-308
[18] Freimanis T, Heller K E, Schønecker B, Bildsøe M (2003) Effects of postnatal stress on the development of type 1 diabetes in bank voles (Clethrionomys glareolus). Int J Exp Diabesity Res 4: 21-25
[19] Samsioe A, Feinstein R, Saade G, et al. (2006) Intrauterine death, fetal malformation, and delayed pregnancy in Ljungan virus-infected mice. Birth Defects Res B Dev Reprod Toxicol 77: 251-256
[20] Blixt M, Niklasson B, Sandler S (2007) Characterization of beta-cell function of pancreatic islets isolated from bank voles developing glucose intolerance/diabetes: an animal model showing features of both type 1 and type 2 diabetes mellitus, and a possible role of the Ljungan virus. Gen Comp Endocrinol 154: 41-47
[21] Maruyama T, Takei I, Asaba Y, et al. (1989) Insulin autoantibodies in mouse models of insulin-dependent diabetes. Diabetes Res 11: 61-65
[22] Utsugi T, Kanda T, Tajima Y, et al. (1992) A new animal model of non-insulin-dependent diabetes mellitus induced by the NDK25 variant of encephalomyocarditis virus. Diabetes Res 20: 109-119
[23] van der Wert N, Hillebrands J L, Klatter F A, Bos I, Bruggeman C A, Rozing J (2003) Cytomegalovirus infection modulates cellular immunity in an experimental model for autoimmune diabetes. Clin Dev Immunol 10: 153-160

What is claimed is:

1. A method of prophylactic and/or therapeutic treatment of a mammal for a disease that is caused by a Ljungan virus infection, comprising administering to said mammal an antivirally effective amount of ribavirin or a derivative thereof effective against the Ljungan virus, and pleconaril or a derivative thereof effective against the Ljungan virus, and eliminating or inhibiting said virus in said mammal and at the same time preventing and/or treating said disease in said mammal.

2. The method according to claim 1, wherein the mammal is selected from the group consisting of humans, horses, cattle, pigs, cats, dogs and rodents.

3. The method according to claim 1, wherein the Ljungan virus infection is in at least one of muscle tissues, neural cells and endocrine glands of the mammal.

4. The method according to claim 3, wherein the muscle tissue is heart tissue, the neural cells are brain cells, the endocrine glands are beta cells of pancreas, thyroid gland and/or supra renal gland.

5. The method according to claim 1, wherein the disease is selected from the group consisting of myocarditis, cardiomyopathia, Guillain Barré syndrome, diabetes mellitus, multiple sclerosis, chronic fatigue syndrome, myasthenia gravis, amyothrophic lateral sclerosis, dermatomyositis, polymyositis, malformation such as anencephaly and hydrocephaly, spontaneous abortion, intrauterine fetal death, pre-eclampsia, lethal central nervous disease, sudden infant death syndrome, Bell's (facial) paralysis, Addison's disease and pernicious anemia.

6. The method of claim 5, wherein the disease is selected from diabetes mellitus and amyothrophic lateral sclerosis.

7. The method according to claim 1, further comprising administering an anti-Ljungan virus antibody.

8. The method according to claim 1, further comprising administering an interferon.

9. A method of prophylactic and/or therapeutic treatment of a mammal for a disease that is caused by a Ljungan virus infection, comprising administering to said mammal a composition comprising ribavirin or a derivative thereof effective against the Ljungan virus, said mammal receiving pleconaril or a derivative thereof effective against the Ljungan virus, and said administering step occurring simultaneously or sequentially with the receiving pleconaril or a derivative thereof.

10. A method of prophylactic and/or therapeutic treatment of a mammal for a disease that is caused by a Ljungan virus infection, comprising administering to said mammal a composition comprising pleconaril or a derivative thereof effective against the Ljungan virus, said mammal receiving ribavirin or a derivative thereof effective against the Ljungan virus, and said administering step occurring simultaneously or sequentially with the receiving ribavirin or a derivative thereof.

11. A composition comprising ribavirin or a derivative thereof effective against a Ljungan virus and pleconaril or a derivative thereof effective against a Ljungan virus.

12. The composition according to claim 11, further comprising an anti-Ljungan virus antibody.

13. The composition according to claim 11, further comprising an interferon.

14. The composition according to any claim 11, effective in therapy.

15. The composition according to claim 11, effective in treating a disease in a mammal that is caused by a Ljungan virus infection.

16. The composition according to claim 15, wherein the disease is selected from the group consisting of myocarditis, cardiomyopathia, Guillain Barré syndrome, diabetes mellitus, multiple sclerosis, chronic fatigue syndrome, myasthenia gravis, amyothrophic lateral sclerosis, dermatomyositis, polymyositis, malformation such as anencephaly and hydrocephaly, spontaneous abortion, intrauterine fetal death, pre-eclampsia, lethal central nervous disease, sudden infant death syndrome, Bell's (facial) paralysis, Addison's disease and pernicious anemia.

17. The composition according to claim 16, wherein the disease is selected from diabetes mellitus and amyothrophic lateral sclerosis.

18. A kit comprising a first pharmaceutical composition comprising ribavirin or a derivative thereof effective against a Ljungan virus and a second pharmaceutical composition comprising pleconaril or a derivative thereof effective against a Ljungan virus, the first and second pharmaceutical compositions being suitable for simultaneous or sequential administration for the treatment of a disease that is caused by a Ljungan virus infection.

19. The kit according to claim 18, further comprising a third pharmaceutical composition comprising an anti-Ljungan virus antibody.

20. The kit according to claim 18, further comprising a pharmaceutical composition comprising an interferon.

21. A method of making a medicament for treatment of a disease in a mammal that is caused by a Ljungan virus infection by combining ribavirin or a derivative thereof effective against a Ljungan virus and pleconaril or a derivative thereof effective against a Ljungan virus into a medicament.

22. The method according to claim 21, further including the step of combining an anti-Ljungan virus antibody in the medicament.

23. The method according to claim 21, further including the step of combining an interferon in the medicament.

24. A method of prophylactic and/or therapeutic treatment of a mammal for a disease that is caused by a Ljungan virus infection, comprising administering to said mammal of an antivirally effective amount of ribavirin or a derivative thereof effective against the Ljungan virus, eliminating or inhibiting proliferation of said virus in said mammal and at the same time preventing and/or treating said disease in said mammal.

25. The method of claim 24, wherein the mammal is selected from the group consisting of humans, horses, cattle, pigs, cats, dogs and rodents such as rats, mice and bank voles.

26. The method of claim 24, wherein the Ljungan virus infection is in at least one of muscle tissues, neural cells and endocrine glands of the mammal.

27. The method of claim 26, wherein the muscle tissue is heart tissue, the neural cells are brain cells, the endocrine glands are beta cells of pancreas, thyroid gland and/or supra renal gland.

28. The method of claim 24, wherein the disease is selected from the group consisting of myocarditis, cardiomyopathia, Guillain Barré syndrome, diabetes mellitus, multiple sclerosis, chronic fatigue syndrome, myasthenia gravis, amyothrophic lateral sclerosis, dermatomyositis, polymyositis, malformation such as anencephaly and hydrocephaly, spontaneous abortion, intrauterine fetal death, pre-eclampsia, lethal central nervous disease, sudden infant death syndrome, Bell's (facial) paralysis, Addison's disease and pernicious anemia.

29. The method of claim 28, wherein the disease is selected from diabetes mellitus and amyothrophic lateral sclerosis.

30. The method of claim 24, further comprising administering an anti-Ljungan virus antibody.

31. The method of claim 24, further comprising administering an interferon.

32. A composition comprising: a) ribavirin or a derivative thereof effective against a Ljungan virus; and b) one or more agents selected from the group consisting of an anti-Ljungan virus antibody and an interferon.

33. A kit comprising a first pharmaceutical composition comprising ribavirin or a derivative thereof effective against a Ljungan virus and a second pharmaceutical composition comprising an anti-Ljungan virus antibody, the first and second pharmaceutical compositions being suitable for simultaneous or sequential administration for the treatment of a disease that is caused by a Ljungan virus infection.

34. A kit comprising a first pharmaceutical composition comprising ribavirin or a derivative thereof effective against a Ljungan virus and a second pharmaceutical composition comprising an interferon, the first and second pharmaceutical compositions being suitable for simultaneous or sequential administration for the treatment of a disease that is caused by a Ljungan virus infection.

35. A kit comprising a first pharmaceutical composition comprising ribavirin or a derivative thereof effective against a Ljungan virus, a second pharmaceutical composition comprising an interferon, and a third pharmaceutical composition comprising an anti-Ljungan virus antibody, the first, second and third pharmaceutical compositions being suitable for simultaneous or sequential administration for the treatment of a disease that is caused by a Ljungan virus infection.
